(19) United States
(12) Patent Application Publication
(10) Pub. No.: US 2004/0082776 A1
(43) Pub. Date: Apr. 29, 2004

(54) Process for recovery of uridine from molasses

(76) Inventors: Matthias Busse, Schulenrode (DE); Hans-Jurgen Danneel, Kalletal (DE); Robert Faurie, Konigslutter (DE)

Correspondence Address: Michele J. Young, Salter & Michaelson, 321 South Main Street, Providence, RI, 02903-7128, US

(21) Appl. No.: 10/654,644

(22) Filed: Sep. 3, 2003

(30) Foreign Application Priority Data
Sep. 3, 2002 (DE) 102 41 116.6-44

(51) Int. Cl. C07H 19/067

(52) U.S. Cl. 536/28.53

(57) ABSTRACT

The disclosure concerns a process for the recovery of uridine from molasses by means of chromatographic processes, whereby uridine is enriched to a high yield and high purity and in particular that uracil is separated from uridine.

### BACKGROUND

1. Technical Field

The disclosure concerns a process for the recovery of uridine from molasses based on chromatographic methods.

2. Related Art

Molasses occurs during sugar recovery from sugar beets as a highly viscous sticky syrup due to its high dry matter content. Besides its main component of sucrose, this dry matter component consists of a number of valuable plant-derived substances.

Examples of these are other saccharides such as oligosaccharides and non-sugar components such as vitamins and organic nitrogen compounds such as amino acids and nucleotides, with their respective building blocks, the nucleosides and nucleobases.

Many of these components in molasses constitute only a small portion of less than 1%, as so-called “minor components.” Reinefeld E. et al ascribe to uridine in molasses a content of 630 mg/dm³ or 882 mg/kg (E. Reinefeld et al. “Zum Nachweis und Vorkommen von Nukleotid-Spaltprodukten in technischen Zuckerlösungen—Beitrag zur Vervollständigung der erfaβten Stickstoffverbindungen” in Zuckerind. 111 (1986) no. 11, pages 1017 to 1024).

These components of molasses are of interest for their function as raw materials and active ingredients, particularly for pharmaceutical purposes and also for human nourishment. For example, the therapeutic benefit of the nucleic acid building block uridine has been demonstrated in clinical studies for various ailments such as coronary disease and hypertension, disease of the respiratory tracts, liver malfunction, sterility, cancer, aids, epilepsy, Parkinson's disease, fear neuroses, sleep disturbances, ischemia and hypoxia. The ability of uridine and some of its derivatives to enter the intracellular milieu of a cell opens a wide future area of pharmaceutical applications (G. P. O'Connell et al. “Uridine and its nucleosides: Biological Actions, Therapeutic Potentials” in Trends in Pharmacol. Sci. 20 (1969), pages 218 to 225, as well as e.g. WO 99/08686, EP 0 348 360 and KR 9408033). In addition, uridine is also of interest as a food supplement, particularly for baby and sportsman nourishment.

Due to its enormous potential applications, there exists a rising demand for uridine. Known processes for the recovery of ribo-nucleosides such as uridine are hydrolysis of RNA, which takes place chemically as in DE 824206, but more often enzymatically by the use of 5′-phospho-diesterase (U.S. Pat. No. 3,304,238). The recovery of nucleosides and nucleotides through hydrolysis comprises the provision of a ribonucleic acid-containing raw material, the recovery of RNA from that, hydrolysis of the RNA and the separation of the building blocks resulting from the hydrolysis. Because the enzyme 5′-phospho-diesterase exhibits non-selective activity towards RNA as well as DNA, at least eight related substances have to be very laboriously separated to make the recovery possible.

Further, fermentation processes (U.S. Pat. No. 4,880,736 and JP 4252190) as well as a bio-transformation process (JP 1074998) have been developed for the recovery of uridine. Whereas the resources input of these for product purification is lower than for the RNA hydrolysis, a higher resources input is needed for the uridine synthesis.

Molasses as a possible source for the raw and active materials which it contains attracted attention, since large quantities of molasses are processed through separation of the high sugar component as liquid sugar for the food industry. The separation of the sucrose fraction from the remaining molasses components normally proceeds through ion exclusion chromatography on strongly acidic cation exchangers (U.S. Pat. Nos. 3,884,714; 4,412,866; 5,795,398). In these processes byproduct fractions occur, in which the molasses-containing substances can be enriched, dependent upon the influences exerted upon them. Yet the fractions obtained from the chromatographic molasses desugarization usually contain a mixture of individual molasses components which have to be further purified to in order to isolate a desired target substance. The composition of the fractions obtained can vary considerably depending on the separation conditions in the individual separation columns as well as due to strong variations in the composition of the molasses.

Oikawa Sh. et al. describe in “Chromatographic Separation of Molasses constituents (Part 7)”, Research Society of Japan Sugar refineries Technologists, Vol.31, 1982, p.55 to 63, the recovery of adenosine, a nucleoside, from clear juice, an intermediate product in sugar recovery. From the concentrate of thin juice, sugar is recovered through crystallization, whereby molasses appears as a byproduct. According to Oikawa, the treatment of thin juice can yield uridine in the basic eluate of the regeneration from the anion exchange resin, when a presumed known combination of cation exchanger and anion exchanger are used for desalination. A chromatographic separation of uridine, particularly of uracil, does however not take place.

Due to their very similar behaviors, the isolation of the individual nucleic acid building blocks such as nucleotides, nucleosides and nucleobases out of the fractions obtained is problematic. In particular, the separation of the nucleosides from the nucleobases was found to be difficult.

Investigations into the separation of the nucleic acids building blocks are recorded by Reinefeld (mentioned above) and J. B. Stark et al. “[t]he purines, pyrimidines and nucleosides in beet diffusion juice and molasses,” in J.Amer.Soc.Sugar Beet Technol. 9, p.201-206. Reinefeld suggests making use of the higher acid strength of the nucleotides for the separation of the nucleosides and nucleobases. Thereby all three components are tied to one strongly basic anion exchanger and the nucleobases are stripped from the nucleobases by means of 2N acetic acid. Thereby the nucleobases and nucleosides will allow themselves to be eluted from the exchanger resin, but not the nucleotides, which will remain tied to the resin. For further separation the eluate, with 2N acetic acid, which contains the nucleobases and nucleosides, is passed onto a strongly acidic cation exchanger (H⁺-form). This will not result in any separation into nucleosides and nucleobases, but merely in a separation corresponding to the bases into purine bases and nucleosides with purine bases as well as into cytidine and cyostine as well as into pyrimidine bases and nucleosides with pyrimidine bases.

A separation of nucleosides and nucleobases, particularly of uridine and uracil, does not take place.

Stark suggested a pre-separation on a cation exchanger (Dowex-50 in H⁺-form) of the purine, pyrimidine and nucleosides contained in the molasses, and subsequently a further separation of the resulting fractions on anion exchangers. For elution of the cation exchangers water and then 0.4N ammonium hydroxide are used, whereby uridine together with uracil and other components are obtained in the water fraction. The further separation of the water fraction is over Permutit A, an anion exchanger, for which water and then a solution saturated with carbon dioxide are used for elution. The purine and pyridine end in the carbon dioxide eluate, whereas the water fraction contains sugar and other neutral substances. A further isolation into individual components does not take place.

The claimant annually separates large quantities of sugar beet molasses chromatographically and has for some time been occupied with the possibility of using molasses as a source for uridine.

A general description of one of the processes used by the claimant for these purposes is given by F. Irtel in his doctoral work “Neue Analytik fur industrielle Aufarbeitungsprozesse”, University of Hanover, 2001, pages 79 to 89. The subject of the doctoral work is basically about the development of biosensors, which show a high selectivity for specific components of molasses, among others uridine, with the object that such biosensors should be utilized for the determination and monitoring of the required separation times of the fractions containing these components which have been obtained during chromatographic separation. In this regard a short description of a chromatographic processing of molasses for recovery of uridine will be given, without closer details.

As is usual in the separation of sucrose during molasses desugarization, the molasses is subjected to ion exclusion chromatography, whereby a generally well-known mono-dispersed sulfonated polystyrene-divinylbenzene resin and water as elution medium are used. The individual fractions from the ion exclusion chromatography are subsequently passed over an anion exchanger, whereby it is noted that a uridine-enriched fraction is obtained, dependent on the elution medium. No further information on the elution media, particularly concrete examples, are available. In a next stage, chromatography takes place over a so-called exchange rack for the separation of impurities such as ash and components which are responsible for the characteristic brown color of molasses. After concentration of the uridine fraction through evaporation and further purification through decolorization and filtration, a further enrichment of uridine by ion exclusion chromatography is performed prior to crystallization.

Neither are instructions for the separation of uracil from uridine given, nor is any indication available on the actual enrichment achieved for uridine. In this regard it is merely noted in summary that, at the point of the anion exchange chromatography the uridine content amounts to 1-10 g/L.

For the utilization of the recovered uridine, particularly in the field of pharmaceuticals, a high purity is an absolute requirement. Investigations into uridine crystallization have shown that, already at a uracil content of only 2 g/100 g uridine, the crystals thus obtained contain uracil at concentrations beyond tolerance.

Besides a high purity, uridine should be obtainable in a commercial process at high yields and at favorable cost. For an economically feasible process sequence, uridine should already be highly enriched in the earlier process stages, so that the process expenditure can be reduced.

### SUMMARY

The present disclosure is directed to a process for the recovery of uridine using molasses or a uridine-containing molasses fraction as starting material. The starting material is passed over a first strongly basic anion exchanger, whereby the bound uridine is eluted at the first anion exchanger. The resulting uridine-containing eluate is subjected to ion exclusion chromatography on a strongly acidic cation exchanger as separation medium. In some embodiments, the salt content of the molasses or the uridine-containing molasses fraction is reduced before feeding to the first anion exchanger.

In another embodiment, the disclosure is directed to a process for the separation of nucleobases from nucleosides, wherein the separation is achieved by ion exclusion chromatography with a strongly acidic cation exchanger.

In another embodiment, the disclosure is directed to the use of uridine alone or in combination with one or more active ingredients in cosmetic preparations, as additive to animal feed or as food additive.

### DETAILED DESCRIPTION OF THE PREFERRED EMBODIMENTS

A process disclosure is made available through which uridine can economically be obtained at high yield and high purity from molasses or a uridine-containing molasses fraction. In particular, the disclosed process enables a separation of the corresponding nucleobase uracil from the uridine fraction.

Further, an economical process for uridine recovery should enable the recovery to be over simple separation stages, without the need for expensive or elaborate specific syntheses or separations.

According to the disclosure, this problem is solved through a process, according to which the molasses or a uridine-containing molasses fraction is passed over a first strongly basic anion exchanger as a first stage, whereby the anion exchanger binds the uridine, a uridine-containing fraction is eluted from the exchanger, and the thus obtained uridine-containing eluate is subjected to ion exclusion chromatography on a strongly acidic cation exchanger.

In accordance with a further aspect, the disclosure relates to a process for the separation of nucleosides from the corresponding nucleobases by means of ion exclusion chromatography.

For the purposes of the disclosure, the division into strong/weak, acidic/basic cation/anion exchanger takes place according to the criteria which are generally recognized in the applicable technical world.

For the process for the recovery of uridine, molasses is utilized as starting material; a raw material in which uridine exists in free form.

For the process according to the disclosure, any uridine-containing molasses fraction can, in principle, be used. If the salt content of the molasses fraction which is used as starting material too high, the salt content in the molasses fraction is preferably reduced before being treated on the anion exchanger, because very high salt contents require an increased amount of anion exchange material and thereby adversely affect the economics. The salt reduction can be achieved by choosing any of the already known methods.

Preferably, a uridine-containing molasses fraction should be used, if necessary with its salt content reduced, such as would be obtained from the well-known molasses desugarization through ion exclusion chromatography on strongly acidic cation exchangers.

It has been shown that uridine, under such conditions, will be enriched to about 200 mg/100 g dry matter in the sucrose fraction, so that the sucrose fraction obtained from the usual molasses desugarization should preferably be used as a starting material for the process in accordance with the disclosure.

Optionally, the process according to the disclosure could include ion exclusion chromatography as a pre-stage, operated according to the practice of molasses desugerization, as a first enrichment of uridine in a suitable fraction.

Besides uridine, the molasses or the uridine-containing molasses fraction contain a range of other components, which include uracil.

For the further enrichment of uridine and stripping of the other substances the desired uridine-containing starting material is subjected to an ion exchange process on a first anion exchanger. Due to its weakly acidic character, arising from the dissociation of the imide, uridine will bind to the anion exchanger.

On the grounds of their high stability against organic fouling, suitable anion exchangers would include macroporous polyacrylate, which contains quaternary amine as functional group. Examples of this are anion exchangers, sold under designations Purolite A 860 S or Amberlite IRA 958. The anion exchanger will be in the OH⁻-form.

In principle, any desired elution medium can be used which will release uridine from the exchanger. Suitable elution media are bases and acids. It has however been shown that the degree of enrichment of uridine as well as its purity varies with the nature of the chosen elution medium.

If the elution is with a base, such as 5% caustic soda, uridine will appear in the eluate, together with all other adsorbed materials including colorants. The uridine content amounts to 25 g/100 g dry matter.

In contrast, elution with acid leads to a far-going separation of uridine from the other components which had separated from the sugar solution. Surprisingly, it has been shown that, with use of an acid, particularly a dilute acid, the uridine content of the eluate compared with the basic eluate can be considerably increased and that an eluate with a uridine content of about 70 g/100 g dry matter can be obtained. According to the disclosure, “dilute acid” means an acid with a content of 0.2 to 3 equivalents/liter and, in particular, from 0.5 to 1 equivalents/liter.

Examples of suitable acids are sulphuric acid, hydrochloric acid and acetic acid.

In the eluate from dilute acids uridine will appear together with uracil and, in contrast with basic elution, reduced quantities of other molasses components such as molasses colorants, ash-forming salts, amino acids and sucrose.

As already mentioned, a uracil content of only 2 g/100 g uridine during crystallization will lead to crystals which contain uracil in concentrations which are above the acceptable limit for the desired applications.

According to the disclosure it was also found that uracil can be separated from uridine or alternatively that the uracil content can be significantly reduced if the obtained eluate containing uridine and uracil is subjected to ion exclusion chromatography. Besides the separation of the uracil or the reduction in the uracil content, other components which might be present will also be considerably removed.

According to the disclosure, the ion exclusion chromatography results in uracil separation on a strongly acidic cation exchanger.

Particularly good results have been achieved with alkali metal ions from saturated, gel-form cation exchangers on a base of sulfonated polystyrene with a water content from 40 to 65%, particularly from 45 to 60%, and a mean granule size of 200 to 450 μm and a uniformity coefficient of <1.6, particularly 1.2.

The elution runs suitably with water, because the total process is preferably designed around aqueous solutions, as is also the case with molasses desugarization.

It has been shown that, at a pH range of 5 to 8, very good results can be obtained, so that this is the preferred range for the application of ion exclusion chromatography for the separation of uracil from uridine. Due to the particularly simple operational control, it is highly preferable to work in the neutral range.

From the eluate obtained, uridine can be crystallized to a yield of 50% and above, to a purity of at least 96 g/100 g dry matter. Obviously the isolation of the uridine from the eluate can also be achieved by any other suitable method.

If necessary, the process according to the disclosure can be combined with further separation processes, in which the further molasses components obtained in the applicable fractions, such as for example molasses colorants, ash-forming salts, amino acids or oligosaccharides, are fully or partly separated, to thereby relieve the load on the system per the disclosure with its first anion exchanger and ion exclusion chromatography.

For such additional processes, ion exclusion methods also will be preferable.

Thus, the uridine-containing fraction which was intended for the first anion exchanger can, prior to feeding this exchanger, be put onto a cation exchanger. As separation medium for the cation exchanger sulfonated polystyrene can be used. This should be in the H⁺-form, to achieve a simultaneous desalination and decolorization of the fraction. If so desired, the fraction can also be passed over a sequence of such cation exchangers and the anion exchangers disclosed herein.

It has also been found that the uridine-containing eluate from the first anion exchanger should be subjected to a treatment on a strongly acidic cation exchanger, preferably followed by a weakly basic anion exchanger, before going to separation over ion exclusion chromatography. In this way a full desalination of the eluate and thereby relief on the ion exclusion chromatography are achieved. Alternatively or if required also additionally, the treatment with the combination of strongly acidic cation exchanger and/or weakly basic anion exchanger can be fitted in after the ion exclusion chromatography.

Sulfonated polystyrene in H⁺-form can be used as a strongly acidic cation exchanger. Examples of this are cation exchangers sold under the names Amberlite IRA 120 and Levatit S 100. Polymers which are equipped predominantly with tertiary amines as functional groups and appear in free base form, can be used as weakly basic anion exchangers. Preferred weakly basic anion exchangers are macroporous polystyrenes, which are equipped exclusively with tertiary amine functional groups.

Preferably the eluate from the first anion exchanger for the separation of foreign salts and colorants should be passed for so long over the currently discussed combination of strongly acidic cation exchanger followed by a weakly basic anion exchanger until one of the exchangers is no longer able to bind the ions which are to be separated off.

The stripping of uridine from its nucleobase uracil through ion exclusion chromatography, according to the disclosure, is not limited to the system uridine/uracil, but is in principle suitable for stripping of nucleosides from nucleobases.

For example, the operations described herein for implementing an ion exclusion chromatography could be generally applied to separate nucleosides from the corresponding nucleobases.

The disclosure thereby also includes a process for the separation of nucleosides and nucleobases through ion exclusion chromatography.

The isolation of uridine from the uridine-containing fraction of the ion exclusion chromatography according to the disclosure can, in principle, be applied in any manner which is suitable with reference to the above.

Preferably the isolation should be by crystallization. The cooling crystallization process has been shown to be particularly suited. For this a solution is used which will be supersaturated at above 40° C. Preferably the solution should exhibit supersaturation at 30° C. and, more preferably, at less than 20°. The maximum respective cooling rates should not exceed 5 K/h, 2 K/h and 0.5 K/h.

If desired, the crystalline product obtained can be subjected to one or more re-crystallizations to further increase the purity of the uridine.

The highly pure uridine obtained according to the disclosure can be used directly or, if desired, after derivatization, alone or in combination with one or more additional active ingredients.

Known areas of usage include pharmaceutical applications and the use as food additive or supplement, how they are added to foods for technological reasons, as well as the use for nourishment-physiological reasons in nourishment supplements or functional food.

Further to that, due to its regulatory influences on biological systems, uridine is not limited to the above applications, but is eminently suited for cosmetic purposes, as supplement for animal feed and domestic pet food or as food additive.

As with the generally known applications in pharmacology and as food additive, uridine can be used as uridine derivative or, where applicable, in combination with one or more other active substances.

In accordance with a further aspect the disclosure thereby covers the use of uridine and uridine derivatives in cosmetics and as ingredient or supplement for animal feed and domestic pet food.

The following examples serve to elucidate the disclosure.

### WORKING EXAMPLES

### Example 1

105 L from the sucrose fraction from a chromatographic molasses desugarization with 0.04% uridine as well as 16.5% dry matter (DM) are passed over 5.7 L of the anion exchanger Purolite A 860, which is strongly basic in OH⁻-form. The exchanger is washed with 6 L fully desalinated water.

For elution of the uridine 2.5 L of a 4% sulfuric acid are used on the exchanger.

The eluate obtained was divided into 25 equally sized fractions with the aid of a sampling system, whereby all fractions with a DM-content of <1% were combined as product fraction. The product fraction thus obtained amounted to 4 L. This solution showed a DM content of 2.5% as well as a uridine content of 1.72%. With that, the uridine purity was increased from 0.24% to 69%.

### Example 2

336.1 L of a solution obtained analogously to Example 1, with 1.17% uridine and 2% DM is passed over 15 L of cation exchanger Lewatit in H⁺-form, followed by being passed over 16 L weakly basic Amberlite IRA 93 anion exchanger in free base form.

The passed-through liquor was combined and concentrated to 37.4% DM. The concentrate contained uridine at a purity of 71% at a yield of 97%.

The concentrate was heated to 80° C. and treated with 10 g Norit CA1 activated carbon per 100 g DM. Subsequently the carbon was removed at 80° C. using a filter cloth.

### Example 3

1 kg of the filtrate obtained in Example 2 was subjected to ion exclusion chromatography at 80° C. employing 16 L strongly acidic cation exchanger Lewatit MDS 1368 in Na⁺-form.

The chromatographic separation column had an internal diameter of 8 cm. Demineralized water at a flow rate of 8 l/h and at 80° C. was used as elution medium.

The solution processed by the ion exclusion chromatography was fractionated with the aid of a sampling system. The product fraction obtained were cut from a rise in DM of above 7.5% until the fall in DM % to below 7.5%. The separation by means of ion exclusion chromatography was repeated 5 times. The product fractions were combined. The uridine content of the combined product fraction was 85 g/100 g DM. The uridine yield amounted to 70%.

The combined product fraction was concentrated at 70° C. under vacuum to a dry matter content of 62 g/100 g solution and subsequently cooled to 25° C. for crystallization.

The crystals obtained were separated under vacuum on a cloth filter and analyzed. The crystals contained 49% of the recovered uridine at a purity of 99 g/100 g DM.

The mother liquor obtained was again concentrated at 70° C. to 76% DM. After cooling to 40° C. and crystallizing the crystal separation was again over a cloth filter. In this after-crystallization 43% of the uridine which remained behind in the mother liquor of the first crystallization was recovered at a purity of 95 g/100 g DM.

### Comparative Example

A solution containing uridine at a content of 83.6 g uridine/100 g solution was prepared analogously to Examples 1 and 2. The solution obtained was immediately subjected to crystallization. Hereby the solution was concentrated under vacuum at 70° C. for direct crystallization to a gross DM content of 81.8%. For crystallization 96 g of the solution was cooled to 30° C. The crystals obtained were separated by sieve centrifuge and analyzed. The crystals contained 46% of the input uridine quantity at a purity of 86 g/100 g DM.

While this disclosure has been particularly shown and described with references to preferred embodiments thereof, it will be understood by those skilled in the art that various changes in form and details may be made therein without departing from the spirit and scope of the disclosure as defined by the appended claims.

What is claimed is:

1. A process for the recovery of uridine using molasses or a uridine-containing molasses fraction as starting material, wherein the starting material is passed over a first strongly basic anion exchanger, whereby the bound uridine is eluted at the first anion exchanger, and the thus obtained uridine-containing eluate is subjected to ion exclusion chromatography on a strongly acidic cation exchanger as separation medium and, when applicable, the salt content of the molasses or the uridine-containing molasses fraction is reduced before feeding to the first anion exchanger.

2. The process according to claim 1, wherein the uridine-containing molasses fraction is a sucrose fraction which is obtained when molasses is subjected to ion exclusion chromatography for molasses desugarization.

3. The process according to claim 1, wherein the first anion exchanger is a macro-porous polyacrylate with quaternary amine as functional group.

4. The process according to claim 2, wherein the first anion exchanger is a macro-porous polyacrylate with quaternary amine as functional group.

5. The process according to claim 1, wherein for the elution of the uridine from the first anion exchanger an acidic elution medium is to be used.

6. The process according to claim 5, wherein a mineral acid is used as acidic elution medium.

7. The process according to claim 6, wherein the acid content of the mineral acid is within the range of 0.2 to 3 equivalents/liter.

8. The process according to claim 1, wherein the separation medium is a gel-like cation exchanger on a base of sulphonated polystyrene with a water content of 40 to 65%, which is saturated with alkali metal ions.

9. The process according to claim 1, wherein the separation medium has a mean granule size in the range of 200 to 450 μm with a uniformity coefficient of <1.6.

10. The process according to claim 1, wherein uracil is separated from the uridine fraction by ion exclusion chromatography.

11. The process according to claim 1, wherein the isolation of the uridine from the thus obtained uridine-containing fraction occurs by means of cooling crystallization.

12. The process according to claim 1, wherein the process comprises the support of additional separation processes for the removal of foreign substances.

13. The process according to claim 12, wherein, for the separation of foreign salts and colorants, the uridine-containing eluate from the first anion exchanger is passed over a combination of a strongly acidic cation exchanger followed by a weakly basic anion exchanger.

14. The process according to claim 12, wherein the uridine-containing starting material is presented to a cation exchanger prior to feeding to the first anion exchanger.

15. The process according to claim 13, wherein the uridine-containing starting material is presented to a cation exchanger prior to feeding to the first anion exchanger.

16. The process according to claim 13, wherein the sequence of cation and anion exchangers will be passed through at least two times.

17. The process according to claim 14, wherein the sequence of cation and anion exchangers will be passed through at least two times.

18. The process according to claim 15, wherein the sequence of cation and anion exchangers will be passed through at least two times.

19. A process for separation of nucleobases from nucleosides, wherein the separation is achieved by ion exclusion chromatography with a strongly acidic cation exchanger.

20. A use of uridine by itself or in combination with one or more active ingredients in cosmetic preparations, as additive to animal feed or as food additive.

21. A process for the recovery of uridine, comprising the steps of:
providing a uridine-containing molasses fraction as starting material;
passing the starting material over a first strongly basic anion exchanger to elute the uridine from the starting material; and
subjecting the eluate to ion exclusion chromatography on a strongly acidic cation exchanger.

22. The process of claim 21, further comprising the step of:
reducing the salt content of the starting material before feeding to the first anion exchanger.
